(19) United States
(12) Patent Application Publication
(10) Pub. No.: US 2008/0025922 A1
(43) Pub. Date: Jan. 31, 2008

(54) BRONZER COMPOSITION

(76) Inventor: Christopher Marrs, Rancho Palos Verdes, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/755,846

(22) Filed: May 31, 2007

Related U.S. Application Data
(60) Provisional application No. 60/803,561, filed on May 31, 2006.

(51) Int. Cl. A61K 8/02; A61K 8/30; A61Q 19/04

(52) U.S. Cl. 424/47; 424/59

(57) ABSTRACT

Bronzer compositions are provided. They comprise (a) water, (b) at least one water-soluble dye; and (c) at least one water-dispersible or water-soluble film-forming polymer. Optionally, a self-tanning agent such as 1,3-dihydroxyacetone may be present.

The present invention relates to a water-based bronzer composition comprising at least one water-soluble dye and a water dispersible or water-soluble, film-forming polymer. The compositions may be in the form of emulsions or foams and provide excellent rub resistance.

### BACKGROUND OF THE INVENTION

Because of the risks associated with sun tanning such as sunburn, many people use self-tanning compositions or bronzer compositions as a means to either achieve a tan without exposure to the sun, obtain a deeper tan with less exposure to the sun, or to extend the natural life of their suntan.

Bronzer compositions add color to the skin to provide a cosmetic benefit, such as better, healthier skin color. They are also used to aid in the application of sunless tanning agents, such as 1,3-dihydroxylacetone. They provide a visual aid to the user to prevent uneven administration on the skin surface and an immediate darkening effect on the skin.

U.S. Pat. No. 6,214,322 to Castro et al. relates to the use of carmine in self-tanning compositions.

US Published Appln. No. US 2006/0045857 relates to a sunless tanning composition that includes a hydrophobic polymer/hydrophilic polymer adduct.

A problem with conventional bronzer compositions containing water-soluble dyes is their tendency to rub off after application, for instance onto clothes or bedding. They rinse off completely with water.

The present invention relates to water-based bronzer compositions that resist rub off. They contain water, at least one water-soluble dye, and at least one water-dispersible or water-soluble film-forming polymer.

### SUMMARY OF THE INVENTION

The invention provides a bronzer composition comprising: (a) water, (b) at least one water-soluble dye; and (c) at least one water-dispersible or water-soluble film-forming polymer.

### DETAILED DESCRIPTION OF THE INVENTION

The composition contains at least one water-soluble dye. Combinations may be used. Examples include caramel, carmine, fluorescein derivatives, methoxsalen, trioxsalen, carbon black, azo dyes, anthraquinone dyes, blue azulenes, guajazulene, chamuzulene, erythrosin, bengal rose, phloxin, cyanosin, daphinin, eosin G, cosin 10B, Acid Red 51, Red Dye 4, Red Dye 40, Blue Dye 1, Yellow Dye 5, Red Dye 4, Orange Dye 4, Ext. Violet Dye 2, and Ext. Yellow Dye 10. Other dyes are listed on page 1628-30 of the International Cosmetic Ingredient Dictionary and Handbook, eds. Wenninger and McEwen (The Cosmetic, Toiletry, and Fragrance Assoc., Washington, D.C., 7th Edition, 1997) (hereinafter “ICT Handbook”), the contents of which are incorporated herein by reference.

The amount of dye in the composition may vary from about 0.0001 to about 10, preferably about 0.0025 to about 7, weight percent based on the total weight of the composition.

The composition comprises at least one polymer capable of providing the composition with a rub resistance after drying on the skin, such that no or substantially no composition rubs off the skin after it has dried. The polymer may be a water-dispersible or water-soluble film forming polymer. Combinations may be used. Preferably, the polymer is water-dispersible rather than water-soluble.

In one embodiment, the polymer is an acrylates/methacrylates copolymer. In particular, the polymer may be a tetrapolymer comprising methacrylic acid, methyl methacrylate, butyl acrylate, and cetyl-eicosinyl methacrylate, for example the acrylates/C12-22 alkylmethacrylate copolymer commercially sold as Allianz™ OPT from International Specialty Products.

In another embodiment, the polymer is diglycol/CHDM/isophthalates/SIP copolymer, for example commercially sold as AQ 38S by Eastman.

In a further embodiment, the polymer is polyimide-1, for example commercially sold as ISP Aquaflex XL-30 by International Specialty Products.

The amount of polymer used depends on the overall composition. An amount sufficient to impart the desired rub resistance should be employed. Amounts up to about 2 weight percent, or 6 weight percent, or 10 weight percent of the total weight of the composition may be utilized.

The composition may optionally comprise a self-tanning agent, that is, a chemical agent capable of producing or inducing the artificial tanning process of the skin by forming brown pigments in the skin, e.g., through the Maillard reaction reported in Bobin, et al., J. Soc. Cosmet. Chem., 35:265-72 (1984). In particular, the self-tanning agent is an α-hydroxy ketone or aldehyde of the formula: 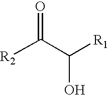
wherein R₁ is H, CH₂OH, CHOHCH₂OH, CH(OH)CH(═O), CH(NH₂)CH(═O), CH(OCH₃)CH(═O), or CH(NH-Phenyl)CH(═O); and R₂ is H or CH₂OH. Examples of such a compound include 1,3-dihydroxyacetone (i.e., dihydroxyacetone) and 1,3,4-trihydroxy-2-butanone (i.e., erythulose). In a preferred embodiment, the self-tanning agent is 1,3-dihydroxyacetone.

The composition advantageously has good resistance to rub off. That is, after applied to the skin, it dries within a relatively short time and does not rub off or substantially does not rub off, for example on clothing or bedding. Tests for rub resistance are well known by those skilled in the art, and such conventional tests for rub resistance of the present composition may be used.

The composition may contain various other optional ingredients known in the art as described below, such as acidifying agents, alkalizing agents, aerosol propellants, antimicrobial agents, antioxidants, buffering agents, chelating agents, coloring additives, dermotologicaly active agents, dispersing agents, emollients, emulsifying agents, humectants, fragrances, preservatives, sugars, sunscreen agents, surfactants, suspending agents, thickening agents, and vehicles. Examples of these ingredients are listed below as well as in the ICT Handbook.

Acidifying and alkalizing agents are added to obtain the desired pH of the composition. Examples of acidifying agents included citric acid, lactic acid, glycolic acid, acetic acid, glacial acetic acid, malic acid, and proprionic acid. Examples of alkalizing agent include edetol, potassium carbonate, potassium hydroxide, sodium borate, sodium carbonate, sodium citrate, sodium lactate, sodium glycolate, and sodium hydroxide. Other acidifying and alkalizing agents are listed on page 1653 of the ICT handbook.

Aerosol propellants are used when the composition is to be administered as an aerosol under pressure. Examples of aerosol propellants include halogenated hydrocarbons such as dichlorodifluoromethane, dichlorotetrafluoroethane, and trichloromonfluoromethane, nitrogen, and volatile hydrocarbons such as butane, propoane, isobutane, or mixtures thereof. Other aerosol propellants are listed on page 1655 of the ICT handbook.

Antimicrobial agents are used when the area that the composition is to be applied is prone to microbial infection, e.g., by bacteria, fungal, or protozoa. Examples of such agents include benzyl alcohol, chlorobutanol, phenylethyl alcohol, phenylmercuric acetate, potassium sorbate, and sorbic acid, benzoic acid, butylparaben, ethylparaben, methylparaben, propylparaben, and sodium benzoate. Other antimicrobial agents are listed on page 1612 of the ICT handbook.

Antioxidants are used to protect ingredients of the composition from oxidizing agents that are included within or come in contact with the composition. Examples of antioxidants include water soluble antioxidants such as ascorbic acid, sodium sulfite, metabisulfite, sodium miosulfite, sodium formaldehyde, sulfoxylate, isoascorbic acid, isoascorbic acid, cysteine hydrochloride, 1,4-diazobicyclo-(2,2,2)-octane, and mixtures thereof. Examples of oil-soluble antioxidants include ascorbyl palmitate, butylated hydroxyanisole, butylated hydroxytoluene, potassium propyl gallate, octyl gallate, dodecyl gallate, phenyl-α-napthyl-amine, and tocopherols such as α-tocopherol. Other agents are listed on pages 1612-13 of the ICT Handbook.

Buffering agents are used to maintain an established pH of the composition. Examples of buffering agents included calcium acetate, potassium metaphosphate, potassium phosphate monobasic, and tataric acid. Other buffering agents are listed on page 1612 of the ICT handbook.

Chelating agents are used to maintain the ionic strength of the composition and/or bind to destructive compounds and metals that are included within or come in contact with the composition. Examples of chelating agents included edatate dipotassium, edetate disodium, edetic acid, and ethylenediamine tetracetic acid (EDTA) and its salts (e.g., tetrasodium EDTA). Other chelating agents are listed on page 1626 of the ICT handbook.

Dermatologically active agents include agents for treating wound healing, inflammation, acne, psoriasis, cutaneous aging, skin cancer, impetigo, herpes, chickenpox, dermatitis, pain, itching, skin irritation. Examples of such dermatologically active agents include hydrocortisone, dexamethesone, panthenol, phenol, tetracycline hydrochloride, yeast, hexylresorcinol, lamin, kinetin, betamethasone, triamcinolone, fluocinolone, methylprednisolone, retnoids such as retinol and retinoic acid, dapsone, sulfasalazine, resorcinol, salicylic acid, benzoyl peroxide, erythromycin-benzoyl peroxide, erythromycin, clindamycin, mupirocin, griseofulvin, azoles such as miconazole, econozole, itraconazole, fluconazole, and ketoconazole, ciclopirox, allylamines such as naftifine and terfinafine, acyclovir, famciclovir, valacyclovir, benzocaine, lidocaine, dibucaine, pramoxine hydrochloride, methyl salicylate, camphor, menthol, resocinol, and vitamins such as tocopherol, and tocopherol acetate.

Examples of dispersing and suspending agents include poligeenan, magnesium aluminum silicate xanthum gum, and silicon dioxide. Other dispersing or suspending agents are listed on page 1612 of the ICT handbook.

Emollients are agents that soften and smooth the skin. Examples of emollients include hydrocarbon oils and waxes such as mineral oil, petrolatum, microcrystalline wax, polyethylene, triglyceride esters such as those of castor oil, cocoa butter, safflower oil, cottonseed oil, corn oil, olive oil, cod liver oil, almond oil, avocado oil, palm oil, sesame oil, squalene, and soybean oil, acetylated monoglycerides, ethoxylated glycerides, fatty acids, alkyl esters of fatty acids, alkenyl esters of fatty acids, fatty alcohols, fatty alcohol ethers, ether-esters, lanolin and derivatives of lanolin, polyhydric alcohol esters, wax esters such as beeswax, vegetable waxes, phospholipids, and sterols. Other emollients are listed on pages 1656-61 of the ICT handbook.

Emulsifying agents are used for preparing the oil-in-water emulsions. Examples of emulsifying agents include Arlacel 165 and methyl gluceth sesquisterate, fatty alcohols, fatty alcohols and alkyl phenols condensed with ethylene oxide. Other emulsifiers are listed on pages 1679-87 of the ICT Handbook. Emulsion stabilizers are listed on pages 1634-35 of the ICT Handbook.

Humectants are agents that promote the retention of moisture, e.g., moisturizers. Examples of humectants include sorbitol, glycerin, glycereth 5 lactate, glycereth 7 triacetate, glycereth 7 diisononoate, hexanetriol, glycols such as methyl-propanediol, 1,2-pentanediol, hexylene glycol, and propylene glycol, alkoxylated glucose, D-panthenol and derivatives thereof, and hyaluronic acid. Other humectants are listed on pages 1661-62 of the ICT Handbook.

Examples of fragrances include peppermint, rose oil, rose water, aloe vera, clove oil, menthol, camphor, eucalyptus oil, and other plant extracts. To eliminate certain odors from compositions, masking agents may be used. An example of a masking agent includes ethylene brassylate. Other fragrances and masking agents are listed on pages 1639-40 of the ICT Handbook.

Preservatives are used to protect the composition from degradation. Examples of preservatives include phenoxyethanol, methylparaben, benzalkonium chloride, benzethonium chloride, propyl paraben, benzoic acid, benzyl alcohol, and mixtures thereof such as liquipar oil. Other preservatives are listed on pages 1654-55 of the ICT Handbook.

Sugars are used to improve the results obtained by the self-tanning agents. Examples of sugars include monosaccharides, disaccharides, and polysaccharides such as glucose, xylose, fructose, reose, ribose, pentose, arabinose, allose, tallose, altrose, mannose, galactose, lactose, sucrose, erythrose, glyceraldehyde, or any combination thereof.

Sunscreen agents are agents used to block or reduce the amount of ultraviolet radiation impinging on the skin (e.g., by absorption, scattering, and reflection of the ultraviolet radiation). Segarin, et al., Cosmetics Science and Technology, Chapter VIII, pages 189, et seq. discloses numerous examples of sunscreen agents. Examples of sunscreen agents include both organic compounds and their salts such as, butylmethoxydibenzoyl methane, diethyl hexyl butamido triazone, diethyl amino hydroxybenzoyl hexyl benzoate, ethylhexyl triazone, bis-ethylhexyloxyphenol methoxyphenyl triazine, methylene bis-benzotriazolyl tetramethylbutyl phenol, phenyl benzymidazole sulfonic acid, ethylhexyl salicylate, benzophenone-3 homosalate, octocrylate, avobenzone, and menthyl anthranilate, as well as inorganic particulate materials such as zinc oxide, silica, iron oxide, titanium dioxide, and 2-ethyl-hexyl-p-methoxycinnamate. Other agents are listed on page 1672 of the ICT Handbook. Generally, the composition may contain from about 1% to about 50%, by weight, of sunscreen agent(s). The exact amounts will vary depending on the sunscreen used and the desired sun-protection factor (SPF).

Surfactants are agents used to stabilize multi-component compositions, e.g., used as wetting agents, antifoam agents, emulsifiers, dispersing agents, and penetrates. Examples of surfactants include lapyrium chloride, laureth 4, laureth 9, monoethanolamine, nonoxynol 4, nonoxynol 9, nonoxynol 10, nonoxynol 15, nonoxynol 30, poloxalene, polyoxyl 8, 40, and 50 stearate, polysorbate 20, polysorbate 40, polysorbate 60, polysorbate 65, polysorbate 80, and polysorbate 85, sodium lauryl sulfate, sorbitan, decyl polyglucoside, PPG-5-ceteth-20 and its derivatives. Other surfactants are listed on pages 1672-90 of the ICT Handbook.

Vehicles are often referred to as the base for the cosmetically acceptable carrier, e.g., a fluid that is capable of delivering the other components of the composition to the skin with acceptable absorption of those components into the skin. Examples of vehicles include water, e.g., deionized water, oil-in-water emulsions, e.g., where the continuous water phase contains the water soluble agents and the discontinuous oil phase contains the oil soluble agents, and water-in-oil emulsions.

In one embodiment, the composition further contains a silicone ingredient. This may impart even greater rub resistance and be particularly useful when the composition is in the form of a foam. Silicone ingredients such as methoxy PEG/PPG-7/3 aminopropyl dimethicone (commercially available as Abil® Soft AF 100) or dimethicone PEG riconoleate (commercially available as Zenicone XX) may be used.

The composition may be in a number of different delivery forms, e.g., a spray, foam, mousse, mist, aerosol, semi-solid cream, liquid such as a solution, emulsion, suspension, lotion, gel, solid such as a powder, adherent stick, flexible mask, or self-hardening liquid or gel, or other suitable forms intended to be applied to the hair, skin, or nails. Water-in-oil emulsions (e.g., ratio of about 10:1 to about 1:100 such as about 1:1 to about 1:10) and oil-in-water emulsions (e.g., ratio of about 10:1 to about 1:100 such as about 1:1 to about 1:10) are typically used in preparing emulsions, lotions and creams.

Preferred are compositions that are in the form or mousses or foams.

In one embodiment, the bronzer composition is alcohol-free.

In another embodiment, the bronzer composition is silicone-free.

The following non-limiting examples further illustrate the invention.

### EXAMPLE 1

A bronzer composition according to the invention was made of the following ingredients. It had excellent rub resistance after drying.

| Ingredient | Amount (weight percent) |
|---|---|
| Water | 86.380 |
| Acrylates/C12-22 alkylmethacrylate | 2.000 |
| copolymer | |
| PVM/MA Decadiene crosspolymer | 0.100 |
| Cetyl hydroxyethylcellulose | 0.020 |
| Sodium hydroxide, 50% solution | 0.060 |
| Sodium citrate | 0.150 |
| Citric acid | 0.050 |
| Carmine | 0.140 |
| Caramel | 3.000 |
| Methyl gluceth-20 | 1.000 |
| Glycerin | 1.000 |
| 1,2-pentadiol | 3.000 |
| 2-methyl-1,3-propanediol | 0.500 |
| Decyl polyglucoside | 1.000 |
| Methylisothiazolinone, methyl paraben | 0.600 |
| polyparaben | |
| PPG-5 Ceteth-20 | 1.000 |

### EXAMPLE 2

A bronzer composition according to the invention was made of the following ingredients. It had excellent rub resistance after drying.

| Ingredient | Amount (weight percent) |
|---|---|
| Water | 84.880 |
| Acrylates/C12-22 alkylmethacrylate | 2.000 |
| copolymer | |
| PVM/MA Decadiene crosspolymer | 0.100 |
| Cetyl hydroxyethylcellulose | 0.020 |
| Sodium hydroxide, 50% solution | 0.060 |
| Sodium citrate | 0.150 |
| Citric acid | 0.050 |
| Carmine | 0.140 |
| Caramel | 4.500 |
| Methyl gluceth-20 | 1.000 |
| Glycerin | 1.000 |
| 1,2-pentadiol | 3.000 |
| 2-methyl-1,3-propanediol | 0.500 |
| Decyl polyglucoside | 1.000 |
| Methylisothiazolinone, methyl paraben | 0.600 |
| polyparaben | |
| PPG-5 Ceteth-20 | 1.000 |

### EXAMPLE 3

A bronzer composition according to the invention was made of the following ingredients. It had excellent rub resistance after drying.

| Ingredient | Amount (weight percent) |
|---|---|
| Water | 84.880 |
| Diglycol/CHDM/Isophthalates/SIP | 2.000 |
| Copolymer | |
| PVM/MA Decadiene crosspolymer | 0.100 |
| Cetyl hydroxyethylcellulose | 0.020 |
| Sodium hydroxide, 50% solution | 0.060 |
| Sodium citrate | 0.150 |
| Citric acid | 0.050 |
| Carmine | 0.140 |
| Caramel | 3.000 |
| Methyl gluceth-20 | 1.000 |
| Glycerin | 1.000 |
| 1,2-pentadiol | 3.000 |
| 2-methyl-1,3-propanediol | 0.500 |
| Decyl polyglucoside | 1.000 |
| Methylisothiazolinone, methyl paraben | 0.600 |
| polyparaben | |
| PPG-5 CETETH-20 | 1.000 |

I claim:

1. A bronzer composition comprising: (a) water, (b) at least one water-soluble dye; and (c) at least one water-dispersible or water-soluble film-forming polymer.

2. The composition of claim 1 further comprising a self-tanning agent having the formula: 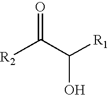
wherein R₁ is H, CH₂OH, CHOHCH₂OH, CH(OH)CH(═O), CH(NH₂)CH(═O), CH(OCH₃)CH(═O), or CH(NH-phenyl)CH(═O); and R₂ is H or CH2OH.

3. The composition of claim 1, wherein the dye is selected from Red Dye 4, Red Dye 40, Blue Dye 1, Yellow Dye 5, Ext. Yellow Dye 10, caramel, carmine, Orange Dye 4, Ext. Violet Dye 2.

4. The composition of claim 1, wherein the polymer is an acrylates/C12-22 alkylmethacrylate copolymer.

5. The composition of claim 1 in the form of a foam.

6. The composition of claim 1 in the form of a mousse.
